(19) United States
(12) Patent Application Publication
(10) Pub. No.: US 2023/0377706 A1
(43) Pub. Date: Nov. 23, 2023

(54) METHODS AND SYSTEMS FOR DYNAMICALLY CUSTOMIZING INSULIN ON BOARD PROFILES AND PROVIDING RECOMMENDATIONS TO IMPROVE THE DURATION OF INSULIN ACTION

(71) Applicant: INSULET CORPORATION, Acton, MA (US)

(72) Inventors: Yibin ZHENG, Hartland, WI (US); Joon Bok LEE, Acton, MA (US); Ashutosh ZADE, San Diego, CA (US); William WHITELEY, Knoxville, TN (US); Jason O’CONNOR, Acton, MA (US)

(21) Appl. No.: 18/199,641

(22) Filed: May 19, 2023

Related U.S. Application Data
(60) Provisional application No. 63/343,739, filed on May 19, 2022.

(51) Int. Cl. G16H 20/10; A61B 5/145; A61B 5/00; G16H 40/63

(52) U.S. Cl. CPC G16H 20/10; A61B 5/14532; A61B 5/4839; G16H 40/63

(57) ABSTRACT

The exemplary embodiments may dynamically adjust an Insulin on Board (JOB) profile for a user of a medicament delivery system to customize the IOB profile to the user based on recent glucose level history and insulin deliveries rather than use the conventional static IOB profiles that are based on population averages. The exemplary embodiments may calculate a Duration of Insulin Action (DIA) for a user from the customized insulin decay curves. The exemplary embodiments may generate and send notifications to the user to help reduce the DIA of the user and/or to keep the DIA of the user from rising. The exemplary embodiments may aim to not overly constrain insulin delivery due to the contribution of insulin boluses to JOB.

### RELATED APPLICATIONS

This application claims the benefit of U.S. Provisional Patent Application No. 63/343,739, filed May 19, 2022, the entire contents of which are incorporated herein by reference in their entirety.

### BACKGROUND

Automated Insulin Delivery (AID) systems deliver small amounts of insulin to users at frequent intervals, such as every 5 minutes. Such AID systems may include control software that determines the size of the insulin doses and then delivers such insulin doses. In some conventional AID systems, the control software seeks to maintain the glucose level of a user at a target glucose level. The control software receives regular glucose level readings from a glucose sensor. The control software predicts the glucose level of the user for a period, such as for the next 12 operation cycles of the delivery device, based on glucose level history and insulin delivery history. Based on these predictions, the control software may choose an insulin delivery dose.

In making the predictions and determining the next insulin delivery dose, the control software may reference the Insulin On Board (JOB) for a user. The IOB refers to the estimated amount of insulin that has not yet acted in the body of the user in that it has not yet affected the glucose level of the user.

Conventional AID systems rely upon IOB curves (sometimes referred to as “insulin decay curves”) for users in predicting future glucose levels of users and determining basal insulin delivery doses. The conventional IOB curves are fixed and based on population averages. FIG. 1 depicts two varieties of conventional IOB curves in a plot 100. The plot 100 shows the fraction of an insulin delivery that remains as IOB over time. The plot 100 shows linear curves 102. The linear curves 102 exhibit linear decay and are available in different decay rates. The second variety of curves 104 depicted in FIG. 1 are known as Ellingsen curves, which are non-linear curves. The Ellingsen curves are also available at different decay rates. An AID system may choose one of these curves at a specified decay rate and use the chosen curve in controlling operation of the AID system.

Conventional AID systems may overly constrain insulin deliveries after large insulin boluses are administered based on the resulting inflated IOB value. As a result, the response by the AID system to rapidly increasing glucose levels may be limited. The control software conventionally must limit its insulin delivery until the IOB returns to a level below the correction insulin amount that would be needed. This constraint fails to account for such large boluses typically being accompanied by user meal ingestion, and thus, the large insulin boluses may already be taken into account within the glucose-insulin dynamics. Hence, it may be sub-optimal to constrain further control software action due to the IOB contributed by meal boluses.

### SUMMARY

In accordance with a first inventive facet, a medicament delivery system for delivering medicament to a user may include a non-transitory readable storage medium storing computer programming instructions and a processor configured for executing the computer programming instructions. The computer programming instructions, when executed, may cause the processor to create a model of analyte-medicament interactions that is customized for the user based on analyte level history and medicament delivery history. The computer programming instructions, when executed, also may cause the processor to determine a standard impulse response for the model, determine, from the standard impulse response, an indication of how much medicament is on board over time for the user after a specified amount of medicament is delivered, and use the indication in determining a dose of medicament for the user to be output by the medicament delivery system.

The medicament delivery system may further include a reservoir for storing the medicament. Exemplary medicaments that may be used include insulin, glucagon-like peptide-1 receptor agonist (GLP-1), glucose-dependent insulinotropic polypeptide (GIP), or other hormones, and/or combinations of medicaments, such as two or more of insulin, GLP-1, and GIP, or other like hormones. This disclosure may refer to “insulin,” “Insulin On Board,” “Duration of Insulin Action,” etc., but it should be understood that the medicaments listed above and others may be used. The analyte level may be a glucose level and/or a ketone level. The indication may be a curve, a function, an equation, a set of equations, a table, or a storage holding values that are indicative of how much medicament is on board for the user over time after the specified amount of medicament is delivered. The computer programming instructions also may cause the processor to initiate delivery of the specified amount of medicament to the user. In some instances, the medicament may be insulin, and the model may be a glucose prediction model that predicts a future glucose level value of the user based on earlier glucose level values and insulin deliveries for the user. The computer programming instructions may further cause updating of the indication to account for recent analyte level values for the user and the updated indication may be used in determining a next dose of medicament for the user to be output by the medicament delivery system. The indication of how much medicament is on board over time for the user after a specified amount of medicament is delivered may be a nonlinear curve.

In accordance with another inventive facet, a medicament delivery system for delivering medicament to a user may include a non-transitory readable storage medium storing computer programming instructions and a processor configured for executing the computer programming instructions. The computer programming instructions may cause the processor to determine an estimate of duration of insulin action for a user, and to compare the estimate of duration of insulin action for the user to a threshold. where the estimate of duration of insulin action (DIA) for the user exceeds a threshold, the computer programming instructions may cause the processor to generate a notification of a recommendation to decrease the duration of insulin action for the user.

The execution of the computer programming instructions by the processor may cause outputting of the notification on a display device. The medicament delivery system may include an on-body medical device, such as medicament delivery device, that is secured to the user, and the recommendation may recommend a change in medicament delivery device infusion location on the user to reduce scarring. The recommendation may recommend a medicament delivery device location on the user that is typically not subject to, or less subject to, pressure by contact.

The recommendation may recommend using faster acting insulin. The recommendation may recommend avoiding ingestion of certain foods, such as certain types of foods or a list of foods.

In accordance with an additional inventive facet, a medicament delivery system for delivering medicament to a user may include a non-transitory readable storage medium storing computer programming instructions and a processor configured for executing the computer programming instructions. Executing the computer programming instructions with the processor may cause the processor to determine an estimate of duration of insulin action for a user, update the estimate of duration of insulin action for the user over time, and where the estimate of duration of insulin action changes more than a threshold amount over time, and generate a notification of a recommendation to reduce the change in the duration of insulin action for the user. As such, the duration of insulin action may be dynamic and automatically adjusted for the user as opposed to a static or fixed or “set” DIA.

Where the estimate of duration of insulin action is increasing, the recommendation may be to change location of where a medicament delivery device that is part of the medicament delivery system is secured to the user. The recommendation may be to modify diet patterns to previous diet patterns that resulted in acceptable glucose level control for the user. The estimate of duration of insulin action may be decreasing, and the recommendation may be to keep a present location of where the medicament delivery device is secured to the user. The medicament delivery device may be an insulin pump adhered to the user.

### BRIEF DESCRIPTION OF THE DRAWINGS

FIG. 1 depicts two varieties of conventional IOB curves in a plot.

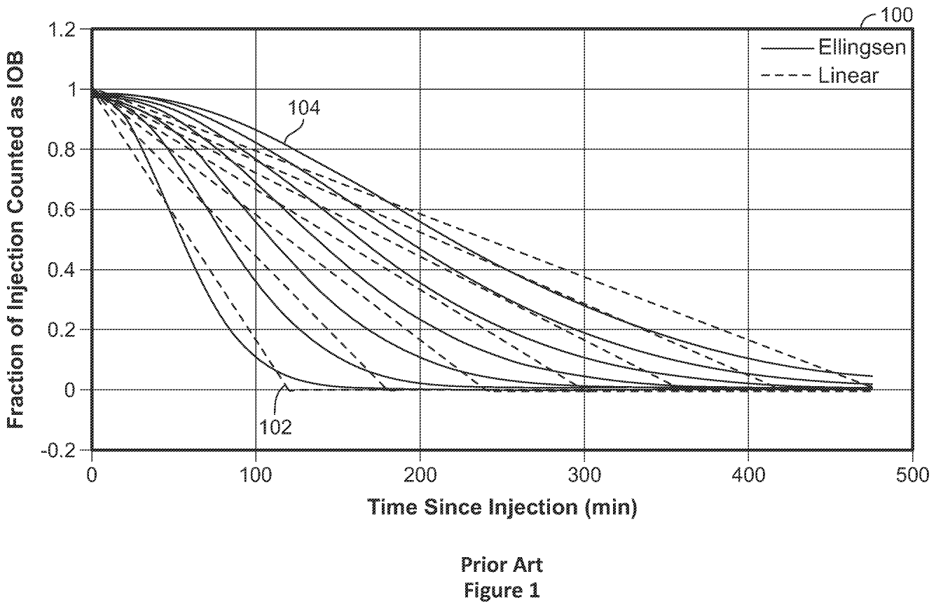

FIG. 2 depicts a block diagram of an illustrative medicament delivery system that is suitable for delivering a medicament to a user in accordance with the exemplary embodiments.

FIG. 3 depicts a flowchart of illustrative steps that may be performed in exemplary embodiments in using customizing and using insulin decay curves in an AID system.

FIG. 4 depicts a flowchart of illustrative steps that may be performed in exemplary embodiments in determining the custom insulin decay curve for the user.

FIG. 5 depicts a flowchart of illustrative steps that may be performed in exemplary embodiments to determine a custom model of insulin-glucose interactions.

FIG. 6 depicts a flowchart of illustrative steps that may be performed in exemplary embodiments in updating the custom model of insulin-glucose interactions.

FIG. 7 depicts examples of trigger events for triggering the model of insulin-glucose interactions in exemplary embodiments.

FIG. 8 depicts a flowchart of illustrative steps that may be performed in exemplary embodiments to account for disturbances when determining the custom model of insulin-glucose interactions.

FIG. 9 depicts a flowchart of illustrative steps that may be performed in exemplary embodiments to create an impulse response per a first option.

FIG. 10 depicts a flowchart of illustrative steps that may be performed in exemplary embodiments to create an impulse response per a second option.

FIG. 11 depicts examples of a plot of impulse response curves.

FIG. 12 depicts a flowchart of illustrative steps that may be performed in exemplary embodiments to convert the impulse response into an insulin decay curve.

FIG. 13 depicts a plot of illustrative impulse response curves and how IOB may be determined from such curves.

FIG. 14 depicts a plot of the resulting insulin decay curves for multiple users per the exemplary embodiments.

FIG. 15 depicts a flowchart of illustrative steps that may be performed in exemplary embodiments to provide recommendations to a user to improve their time in range based upon a DIA for the user.

FIG. 16 depicts a number of example recommendations that may be generated and sent in the exemplary embodiments.

FIG. 17 depicts a flowchart of illustrative steps that may be performed in exemplary embodiments to generate recommendations for certain physiological changes that will improve the time in range for a user based on changes in the DIA of the user.

FIG. 18 depicts some illustrative recommendations for physiological changes in the exemplary embodiments.

FIG. 19 depicts an example recommendation for keeping a location of the medicament delivery device on the user.

FIG. 20 depicts a flowchart of illustrative steps that may be performed in exemplary embodiments to calculate IOB_{bol} from DIA_{bol}(i).

FIG. 21 depicts a flowchart of illustrative steps that may be performed in exemplary embodiments to determine DIA_{bol}(i).

### DETAILED DESCRIPTION

The exemplary embodiments may dynamically and automatically adjust an IOB profile for a user of a medicament delivery device to customize the IOB profile to the user based on recent glucose level history and insulin deliveries rather than on the conventional static IOB profiles that are derived from population averages. This dynamic customization may improve the glucose level management performance of the medicament delivery device for the user by more accurately capturing the IOB of the user over time. The IOB profile for a user is dynamic in that the IOB profile may be automatically updated at regular intervals or responsive to other trigger events.

For a user, a customized IOB profile may be generated as an insulin decay curve in the exemplary embodiments. The process of customizing the IOB profile begins with creating a custom model of insulin-glucose interactions. This model may predict future glucose level values for the user based on past glucose level values and weight coefficients. An impulse response (e.g., a response to a bolus pulse) for the model may be calculated, and the impulse response may be converted into a customized insulin decay curve.

The exemplary embodiments may calculate a DIA for a user from the customized insulin decay curves. Given that there is an association between time in a desired glucose level range and DIA, the exemplary embodiments may compare the DIA for the user with one or more thresholds to determine whether the user can take any steps to reduce the DIA when needed. The exemplary embodiments may generate and output notifications to the user to help reduce the DIA of the user. In addition, the exemplary embodiments may monitor the rate of change of the DIA of the user. If the DIA is increasing over time, recommendations may be generated and output to the user to take actions that will reverse, slow, or halt the increase and/or return to a more acceptable DIA. Where the DIA is decreasing, a recommendation may be generated and sent that recommends continued use of their current choices that affect DIA so as to decrease the DIA, maintain an ideal DIA, or keep the DIA from increasing.

The exemplary embodiments may aim to not overly constrain insulin delivery due to the contribution of insulin boluses to JOB. In some exemplary embodiments, the JOB used by the control software may be partitioned between IOB due to bolus deliveries and IOB due to AID delivery (such as basal insulin deliveries). Thus, the user may have different (simultaneous) Durations of Insulin Action. The IOB due to the bolus deliveries may be based upon a DIA that is particular to insulin boluses. The JOB due to basal deliveries may be based upon a DIA that is particular to basal insulin or AID deliveries of basal insulin. The DIA that is particular to insulin boluses may be shorter than the DIA due to AID delivery to avoid constraining insulin delivery for too long due to the contribution of insulin boluses to JOB. A combined JOB value may be used that reflects the shorter DIA for bolus deliveries and the longer DIA for basal deliveries. In addition, the DIA that is particular to insulin boluses may be dynamically varied based on actual insulin bolus activity. The DIA that is particular to insulin boluses may shorten with increasing cumulative recent insulin bolus deliveries and may increase with decreasing cumulative recent insulin bolus deliveries.

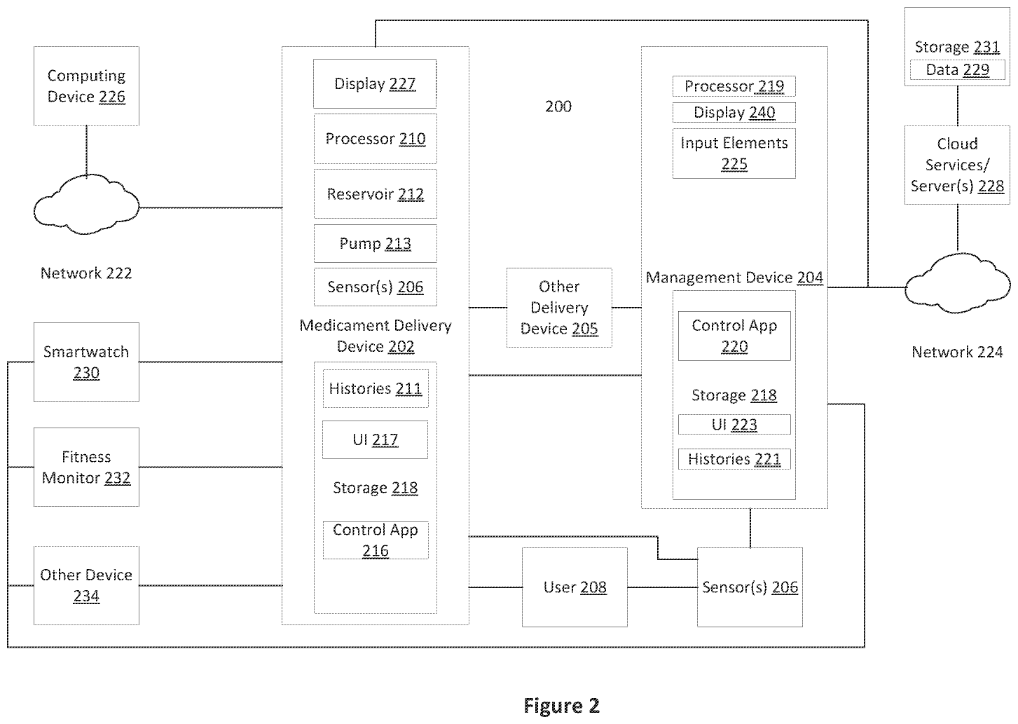

FIG. 2 depicts a block diagram of an illustrative medicament delivery system 200 that is suitable for delivering a medicament, such as insulin and those mentioned above, to a user 208 in accordance with the exemplary embodiments. The medicament delivery system 200 may include a medicament delivery device 202. The medicament delivery device 202 may be a wearable device that is worn on the body of the user 208 or carried by the user. The medicament delivery device 202 may be directly coupled to a user (e.g., directly attached to a body part and/or skin of the user 208 via an adhesive or the like) with no tubes and an infusion location directly under the medicament delivery device 202, or carried by the user (e.g., on a belt or in a pocket) with the medicament delivery device 202 connected to an infusion site where the medicament is injected using a needle and/or cannula. A surface of the medicament delivery device 202 may include an adhesive to facilitate attachment to the user 208.

The medicament delivery device 202 may include a processor 210. The processor 210 may be, for example, a microprocessor, a logic circuit, a field programmable gate array (FPGA), an application specific integrated circuit (ASIC) or a microcontroller. The processor 210 may maintain a date and time as well as other functions (e.g., calculations or the like). The processor 210 may be operable to execute a control application 216 encoded in computer programming instructions stored in the storage 214 that enables the processor 210 to direct operation of the medicament delivery device 202. The control application 216 may be a single program, multiple programs, modules, libraries or the like. The processor 210 also may execute computer programming instructions stored in the storage 214 for a user interface (UI) 217 that may include one or more display screens shown on display 227. The display 227 may display information to the user 208 and, in some instances, may receive input from the user 208, such as when the display 227 is a touchscreen.

The control application 216 may control delivery of the medicament to the user 208 per a control approach like that described herein. The control application may use a glucose prediction model as described below for predicting future glucose levels of the user 208. The storage 214 may hold histories 211 for a user, such as a history of basal deliveries, a history of bolus deliveries, and/or other histories, such as a meal event history, exercise event history, glucose level history, other analyte level history, and/or the like. In addition, the processor 210 may be operable to receive data or information. The storage 214 may include both primary memory and secondary memory. The storage 214 may include random access memory (RAM), read only memory (ROM), optical storage, magnetic storage, removable storage media, solid state storage or the like.

The medicament delivery device 202 may include a tray or cradle and/or one or more housings for housing its various components including a pump 213, a power source (not shown), and a reservoir 212 for storing medicament for delivery to the user 208. A fluid path to the user 208 may be provided, and the medicament delivery device 202 may expel the medicament from the reservoir 212 to deliver the medicament to the user 208 using the pump 213 via the fluid path. The fluid path may, for example, include tubing coupling the medicament delivery device 202 to the user 208 (e.g., tubing coupling a cannula to the reservoir 212), and may include a conduit to a separate infusion site. The medicament delivery device 202 may have operational cycles, such as every 5 minutes, in which basal doses of medicament are calculated and delivered as needed. These steps are repeated for each cycle.

There may be one or more communications links with one or more devices physically separated from the medicament delivery device 202 including, for example, a management device 204 of the user and/or a caregiver of the user, sensor(s) 206, a smartwatch 230, a fitness monitor 232 and/or another variety of device 234. The communication links may include any wired or wireless communication links operating according to any known communications protocol or standard, such as Bluetooth®, Wi-Fi, a near-field communication standard, a cellular standard, or any other wireless protocol.

The medicament delivery device 202 may interface with a network 222 via a wired or wireless communications link. The network 222 may include a local area network (LAN), a wide area network (WAN), a cellular network, a Wi-Fi network, a near field communication network, or a combination thereof. A computing device 226 may be interfaced with the network 222, and the computing device may communicate with the medicament delivery device 202.

The medicament delivery system 200 may include one or more sensor(s) 206 for sensing the levels of one or more analytes. The sensor(s) 206 may be coupled to the user 208 by, for example, adhesive or the like and may provide information or data on one or more medical conditions, physical attributes, or analyte levels of the user 208. The sensor(s) 206 may be physically separate from the medicament delivery device 202 or may be an integrated component thereof. The sensor(s) 206 may include, for example, glucose monitors, such as continuous glucose monitors (CGM's) and/or non-invasive glucose monitors. The sensor(s) 206 may include ketone sensors, other analyte sensors, heart rate monitors, breathing rate monitors, motion sensors, temperature sensors, perspiration sensors, blood pressure sensors, alcohol sensors, or the like. Some sensors 206 may also detect characteristics of components of the medicament delivery device 202. For instance, the sensors 206 in the medicament delivery device may include voltage sensors, current sensors, temperature sensors and the like.

The medicament delivery system 200 may or may not include a management device 204. In some embodiments, no management device is needed as the medicament delivery device 202 may manage itself. The management device 204 may be a special purpose device, such as a dedicated personal diabetes manager (PDM) device. The management device 204 may be a programmed general-purpose device, such as any portable electronic device including, for example, a dedicated controller, such as a processor, a micro-controller, or the like. The management device 204 may be used to program or adjust operation of the medicament delivery device 202 and/or the sensor(s) 206. The management device 204 may be any portable electronic device including, for example, a dedicated device, a smartphone, a smartwatch, or a tablet. In the depicted example, the management device 204 may include a processor 219 and a storage 218. The processor 219 may execute processes to manage a user's glucose levels and to control the delivery of the medicament to the user 208. The medicament delivery device 202 may provide data from the sensors 206 and other data to the management device 204. The data may be stored in the storage 218. The processor 219 may also be operable to execute programming code stored in the storage 218. For example, the storage 218 may be operable to store one or more control applications 220 for execution by the processor 219. Storage 218 may also be operable to store historical information such as medicament delivery information, analyte level information, user input information, output information, or other historical information. The control application 220 may be responsible for controlling the medicament delivery device 202, such as by controlling the automated medicament delivery (ADD) (or, for example, automated insulin delivery (AID)) of medicament to the user 208. The storage 218 may store the control application 220, histories 221 like those described above for the medicament delivery device 202, and other data and/or programs.

A display 240, such as a touchscreen, may be provided for displaying information. The display 240 may display user interface (UI) 223. The display 240 also may be used to receive input, such as when it is a touchscreen. The management device 204 may further include input elements 225, such as a keyboard, button, knobs, or the like, for receiving input form the user 208.

The management device 204 may interface with a network 224, such as a LAN or WAN or combination of such networks, via wired or wireless communication links. The management device 204 may communicate over network 224 with one or more servers or cloud services 228. Data, such as sensor values, may be sent, in some embodiments, for storage and processing from the medicament delivery device 202 directly to the cloud services/server(s) 228 or instead from the management device 204 to the cloud services/server(s) 228.

Other devices, like smartwatch 230, fitness monitor 232 and device 234 may be part of the medicament delivery system 200. These devices 230, 232 and 234 may communicate with the medicament delivery device 202 and/or management device 204 to receive information and/or issue commands to the medicament delivery device 202. These devices 230, 232 and 234 may execute computer programming instructions to perform some of the control functions otherwise performed by processor 210 or processor 219, such as via control applications 216 and 220. These devices 230, 232 and 234 may include displays for displaying information. The displays may show a user interface for providing input by the user, such as to request a change or pause in dosage, or to request, initiate, or confirm delivery of a bolus of medicament, or for displaying output, such as a change in dosage (e.g., of a basal delivery amount) as determined by processor 210 or management device 204. These devices 230, 232 and 234 may also have wireless communication connections with the sensor 206 to directly receive analyte measurement data. Another delivery device 205, such as a medicament delivery pen (such as an insulin pen), may be accounted for (e.g., in determining JOB) or may be provided for also delivering medicament to the user 208.

The functionality described herein for the exemplary embodiments may be under the control of or performed by the control application 216 of the medicament delivery device 202 or the control application 220 of the management device 204. In some embodiments, the functionality wholly or partially may be under the control of or performed by the cloud services/servers 228, the computing device 226 or by the other enumerated devices, including smartwatch 230, fitness monitor 232 or another wearable device 234.

In the closed loop mode, the control application 216, 220 determines the medicament delivery amount for the user 208 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 202 need not deliver one medicament alone. Instead, the medicament delivery device 202 may one medicament, such as insulin, for lowering glucose levels of the user 208 and also deliver another medicament, such as glucagon, for raising glucose levels of the user 208. The medicament delivery device 202 may deliver a glucagon-like peptide (GLP)-1 receptor agonist medicament for lowering glucose or slowing gastric emptying, thereby delaying spikes in glucose after a meal. In other embodiments, the medicament delivery device 202 may deliver pramlintide, or other medicaments that may substitute for insulin. In other embodiments, the medicament delivery device 202 may deliver concentrated insulin. In some embodiments, the medicament or medicament delivered by the medicament delivery device may be a coformulation of two or more of those medicaments identified above. In a preferred embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

As was mentioned above, exemplary embodiments may provide dynamic customized IOB profiles per user. The IOB profiles are customized for a user based on insulin delivery history and glucose level history. The IOB profiles are dynamic in that they may be updated regularly in exemplary embodiments to adapt to the needs of the user.

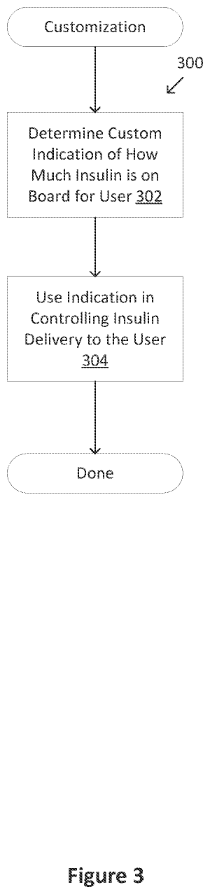

In the exemplary embodiments, the IOB profiles of users may be captured by insulin decay curves that depict the remaining insulin action of a dose of insulin that was delivered to the respective users over time. FIG. 3 depicts a flowchart 300 of illustrative steps that may be performed in exemplary embodiments in such customizing and use of insulin decay curves in an AID system. Initially, at 302, an indication of how much insulin is on board for a user, such as an insulin decay curve, is determined for the user from glucose level history and insulin delivery history. More generally, the indication may be a curve, a function, an equation, a set of equations, a table, or a storage holding values that are indicative of how much medicament is on board for the user over time after the specified amount of medicament is delivered. Since actual data for the user may be used, the indication may be customized for the user. The indication may be used by the AID control system (e.g., control applications 216 or 220) in predicting future glucose levels for the user. These future glucose levels may be used in determining the response of the control system to current and predicted conditions. The determining of the response may include determining the basal insulin delivery dosages for delivery to the user, including suspension of delivery of basal insulin deliveries to the user in some instances.

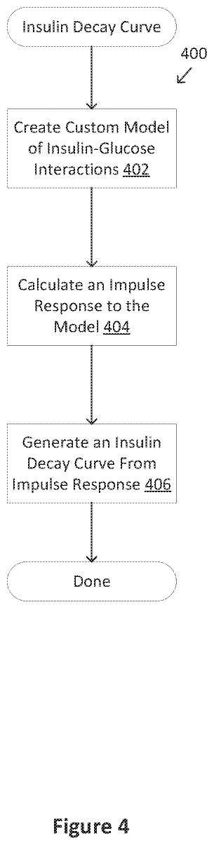

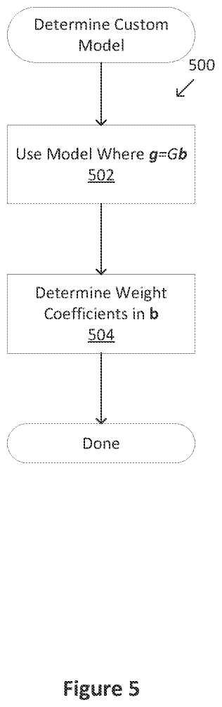

FIG. 4 depicts a flowchart 400 of illustrative steps that may be performed in exemplary embodiments in determining the indication, where the indication is a custom insulin decay curve for the user. At 402, a custom model of insulin-glucose interactions is determined. FIG. 5 depicts a flowchart 500 of illustrative steps that may be performed in exemplary embodiments to determine the custom model of insulin-glucose interactions. The model may be characterized as a recursive model of past glucose level values and past insulin deliveries. For instance, the model may be expressed as:

G_{Δ}(k)=b₁G_{Δ}(k−1)+b₂G_{Δ}(k−2)+b₃G_{Δ}(k−3)−KI_{Δ}(k−3) (Equation 1)

where G_{Δ}(k) is a delta between a predicted glucose level at cycle k and a target glucose level value, b₁, b₂, and b₃ are weight coefficients for the past glucose values, I_{Δ}(k−3) is a delta between the insulin dose delivered at cycle k−3, and K is a weight coefficient. The model can be simplified as:

G_{Δ}(k)=b₁G_{Δ}(k−1)+b₂G_{Δ}(k−2)+b₃G_{Δ}(k−3)+D(k) (Equation 2)

where D(k) is a disturbance at cycle k. The simplification of the model allows the model to be set as a model of only past glucose level values. This can expressed in matrix form g=Gb at 502. The matrices are as follows:

$$[ \begin{matrix}\begin{matrix}\begin{matrix}\begin{matrix}G ( k ) \\ G ( k - 1 )\end{matrix} \\ G ( k - 2 )\end{matrix} \\ ⋮\end{matrix} \\ G ( k - M )\end{matrix} ] = [ \begin{matrix}G ( k - 1 ) & G ( k - 2 ) & G ( k - 3 ) \\ G ( k - 2 ) & G ( k - 3 ) & G ( k - 4 ) \\ G ( k - 3 ) & G ( k - 4 ) & G ( k - 5 ) \\ ⋮ & ⋮ & ⋮ \\ G ( k - M - 1 ) & G ( k - M - 2 ) & G ( k - M - 3 )\end{matrix} ] [ \begin{matrix}\begin{matrix}{b}_{1}^{*} \\ {b}_{2}^{*}\end{matrix} \\ {b}_{3}^{*}\end{matrix} ] . \qquad ( Equation 3 )$$

The parameters in matrix b may then be solved at 504 using the following equation:

b=(G^{T}G)⁻¹G^{T}g (Equation 4).

Thus, the weight coefficients in matrix b are determined.

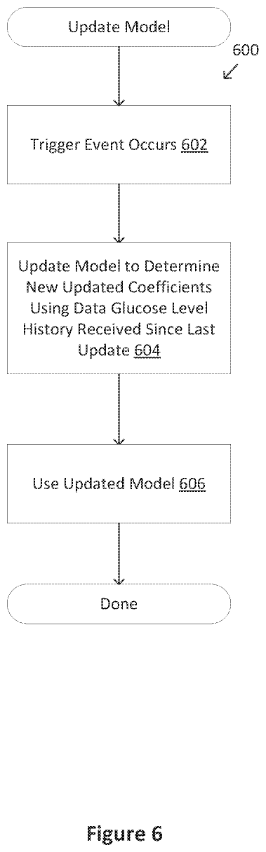

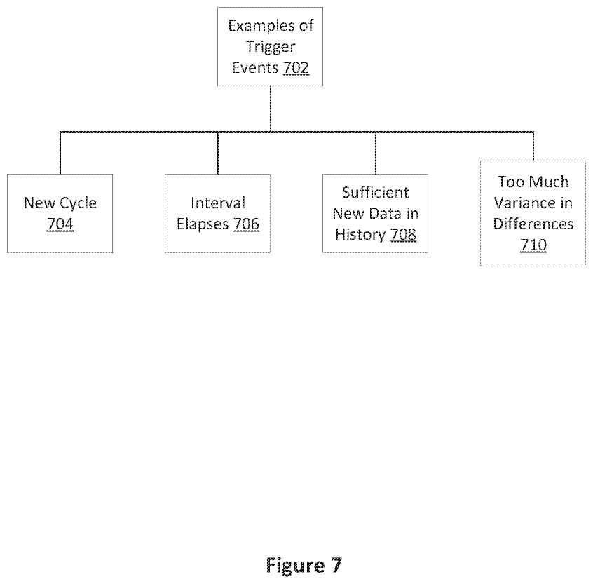

This model of insulin-glucose interactions may be updated. FIG. 6 depicts a flowchart 600 of illustrative steps that may be performed in exemplary embodiments in updating the model. At 602, a trigger event occurs. FIG. 7 depicts examples of such trigger events 702. For instance, a new cycle beginning 704 may act as a trigger to update the model. The elapsing of an interval 706, such as an hour, multiple hours, a day or multiple days (like 3 days), or replacement of a disposable pump device or portion of a pump device (e.g., after 3 or 3.5 days) may act as a trigger event. The presence of sufficient new data 708, such as a sufficient quantity of new glucose level history, may act as a trigger event. The detection of too much variance in the differences between the predicted glucose level values and the actual glucose level values 710 also may act as a trigger. It should be appreciated that combinations of these trigger events 704, 706, 708, and 710 may be used as trigger events. Moreover, other trigger events that are not shown may be used.

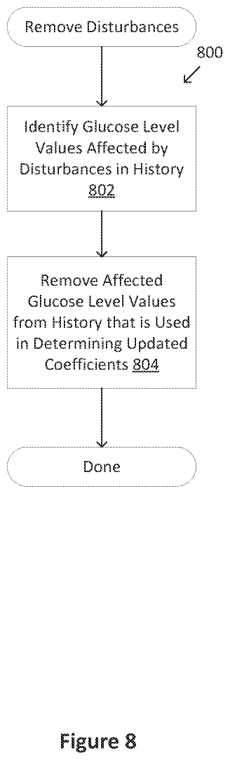

In determining the model, certain data in the glucose level value history may be omitted from consideration. FIG. 8 depicts a flowchart 800 of illustrative steps that may be performed in exemplary embodiments to account for disturbances when determining the model. At 802, glucose level values in the glucose level history that are affected by disturbances may be identified. Examples of disturbances include meals and exercise. The glucose level values affected by the disturbances may be removed from the history that is used in determining the model at 804. The affected glucose level values may be those following meals for a period of time (e.g., 30 min, 1 hour, 2 hours, or 3 hours) or those following the start of exercise for a period (e.g., 30 min, 1 hour, or 2 hours) or those following conclusion of exercise for a period (e.g., 30 min or 1 hour).

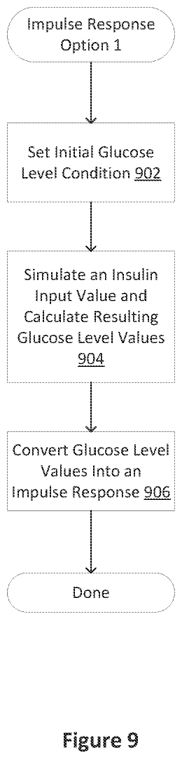

With reference again to FIG. 4, the model with the weight coefficients may then be used to create an impulse response to the model at 404. FIG. 9 depicts a flowchart 900 of illustrative steps that may be performed in exemplary embodiments to create the impulse response per a first option. At 902, an initial glucose level condition is set. At 904, an insulin input value may be simulated and calculations of the predicted glucose level values may be determined using the model. At 906, the predicted glucose level values are converted into an impulse response. An impulse response is the output (e.g., glucose level) produced when the model is presented with the impulse input (e.g., an insulin bolus dose or an insulin basal dose).

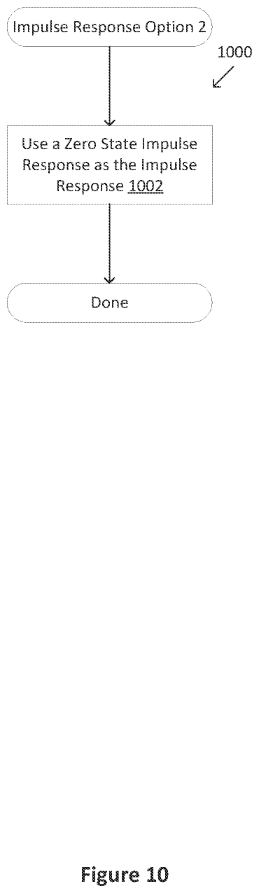

Another option for producing an impulse response for the model with the determined parameters (i.e., weight coefficients) is depicted in the flowchart 1000 of FIG. 10 of illustrative steps that may be performed in exemplary embodiments. At 1002, a zero state impulse response may be calculated. The zero state impulse response is when G(0)=G(1)=G(2)=0, G(3)=K, and for k≥1:

G(k+3)=b₁G(k+2)+b₂G(k+1)+b₃G(k)+Kδ(k) (Equation 4).

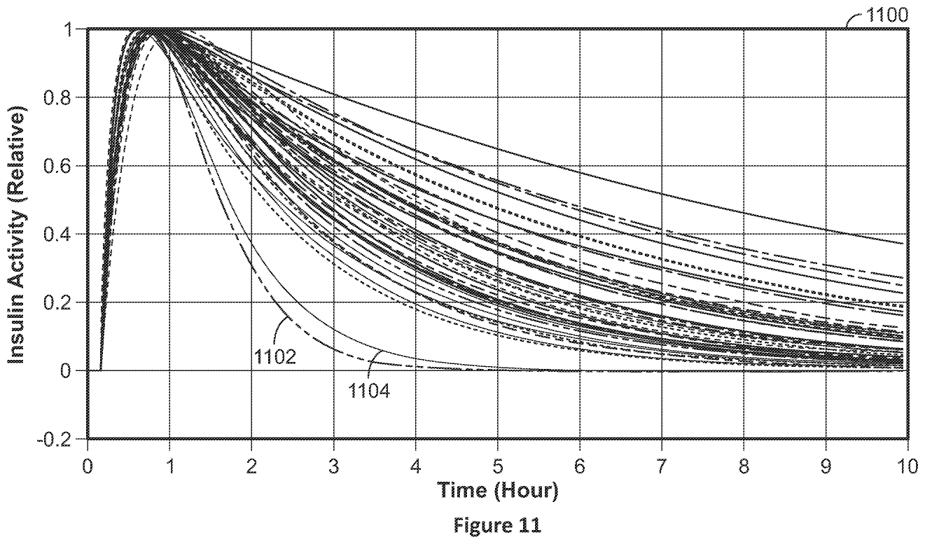

FIG. 11 depicts an example of a plot 1100 of impulse response curves. Each curve is for a different user. Thus, curve 1102 is for a first user, and curve 1104 is for a second user, etc. As can be seen via the impulse response curve, the insulin activity quickly spikes after delivery and then declines over time.

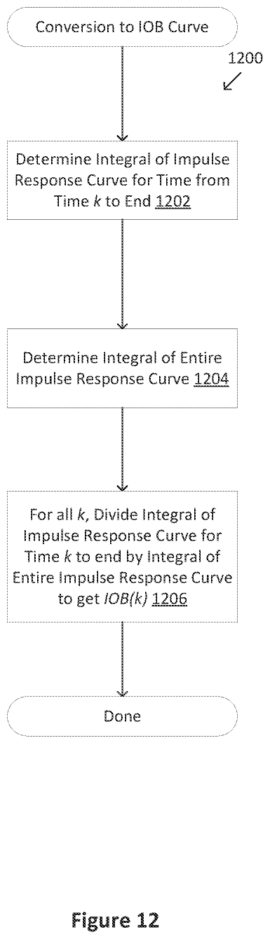

With reference again to FIG. 4, at 406, an insulin decay curve is generated from the impulse response curve for a user. FIG. 12 depicts a flowchart 1200 of illustrative steps that may be performed in exemplary embodiments to convert the impulse response into an IOB curve. The steps depicted are for determining the IOB curve from cycle k forward. At 1202, the integral of the curve from cycle k to the end is determined. This represents the total insulin that remains in the body of the user that still can affect glucose levels of the user. At 1204, the integral of the entire impulse response curve is determined. This represents the entire insulin delivered. At 1206, for all cycles k, the integral of the impulse response curve at cycle k is divided by the integral of the entire response curve to get the JOB for each cycle k so as to produce a normalized insulin decay curve that ranges from 1 to 0.

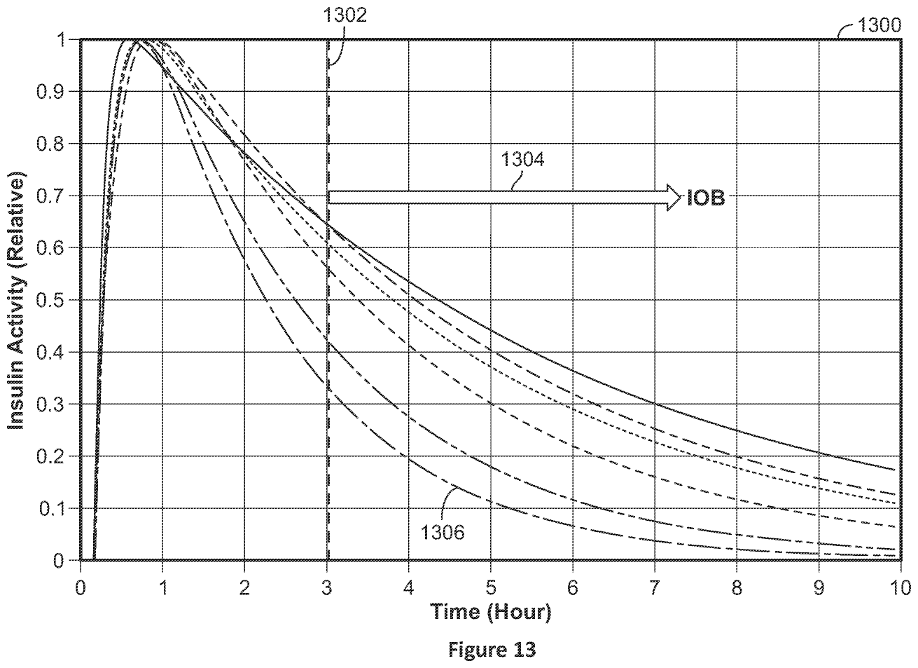

FIG. 13 depicts a plot 1300 of illustrative impulse response curves. At cycle 3, designated by line 1302, the integral of curve 1306 (i.e., the area under the curve) from cycle 3 forward, as indicated by arrow 1304, divided by the integral for the entire curve 1306 is used to determine the insulin decay curve for the user associated with curve 1306. Stated differently, the area under the curve is calculated to assess JOB; area under the curve to the right of line 1302 is compared to the total area under the whole curve (e.g., curve 1306). And if this area under the curve to the right of line 1302 is 30% of the total area under the curve 1306, then there is 30% insulin activity or JOB remaining.

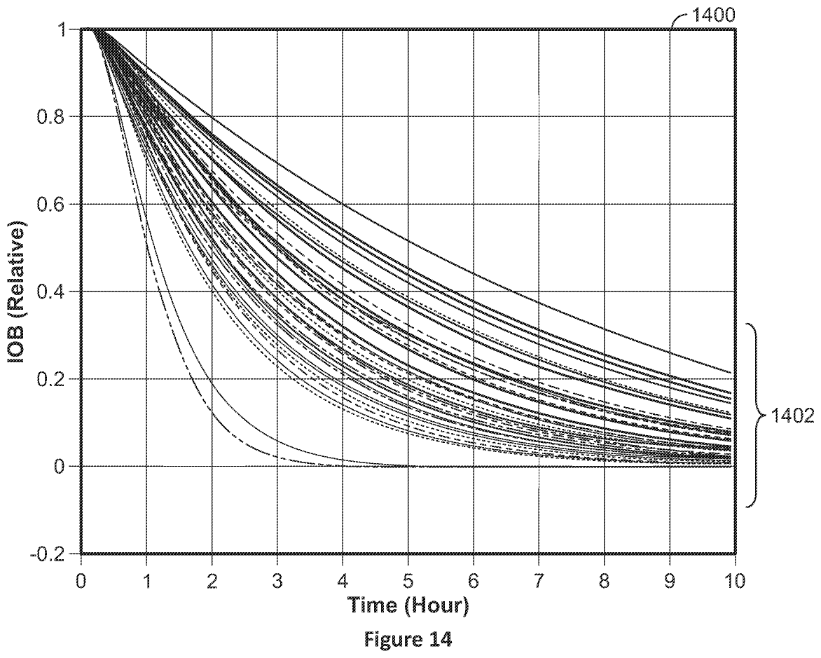

If you plot the remaining area under the curve over time, this results in the graph shown in FIG. 14, representing the JOB curves, or the remaining JOB for an impulse (originally given at time 0) at different times, for different users. Thus, FIG. 14 depicts a plot 1400 of the resulting insulin decay curves 1402 for multiple users. The plot 1400 shows relative JOB over time for the users. Each of the curves 1402 begins at 1 and degrades to 0 over time.

The exemplary embodiments may analyze the resulting customized insulin decay curves to provide recommendations to the user to improve time in range for the user. Time in range denotes the time duration that a glucose level value of the user remains in a desirable range, such as within a defined range surrounding the target glucose level value of the user (e.g., 100 mg/dL to 150 mg/dL). The time in range for a user can be correlated to their DIA. DIA is the time from the injection of insulin until the entire metabolic effect of the insulin has ended (e.g., the time span of the insulin decay curve from injection until 0 insulin action is reached). Shorter DIA is correlated with better time in range and a user's DIA is reflective of their lifestyle and body physiology.

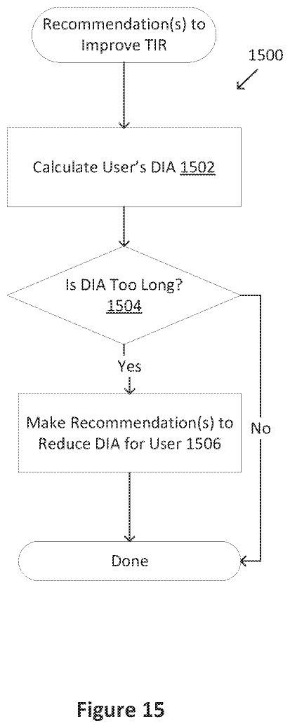

FIG. 15 depicts a flowchart 1500 of illustrative steps that may be performed in exemplary embodiments to provide recommendations to a user to improve their time in range based upon a duration of insulin action (DIA) for the user. At 1502, the DIA for the user is calculated. At 1504, a check is made of whether the DIA for the user is too long, such as by comparing the DIA of the user to a threshold amount. If the DIA is too long, at 1506, one or more recommendations may be provided to the user, as detailed below. The recommendations may take the form of messages conveyed to the user, such as by displaying the messages on display devices 227 and/or 240. Alternately or in combination with the textual messages, audio messages may be output to the user or video messages may be output via the display devices 227 and/or 240. Still further, the notifications may be sent via messaging services, like SMS, or via electronic mail. The recommendations may, in some embodiments, be displayed on web pages that are accessible to the user. If the DIA is not too long (e.g., a user's calculated DIA does not surpass a threshold), a recommendation may not be sent.

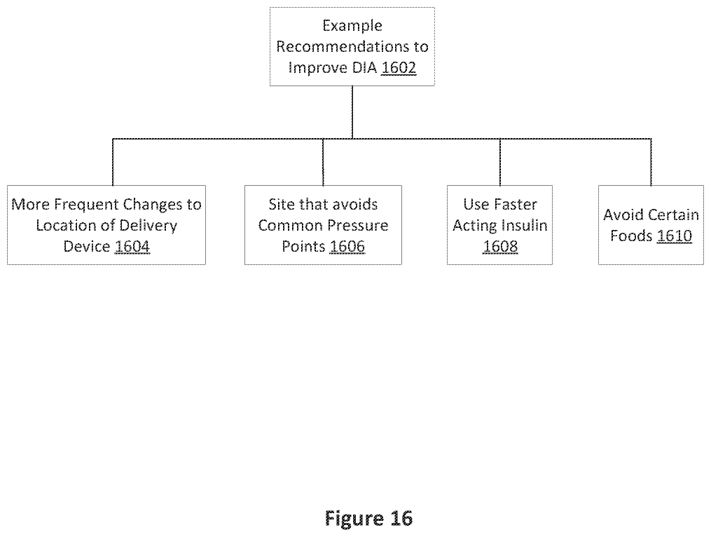

FIG. 16 depicts a number of example recommendations 1602. One example recommendation 1604 is for the user more frequently change the location where the medicament delivery device 202 is attached on the user. If the medicament delivery device 202 remains at or near a single location of the user for too long, the result may be scarring that may affect the DIA as the scarring may make it more difficult for the delivered insulin to be locally absorbed and metabolized. Another recommendation 1606 is avoid sites on the user that are frequently subject to external pressure (“pressure points”), such as on the user's back, which may frequently rest against a seat or bed and thus be subject to pressure. An additional recommendation 1608 may be for the user to use faster acting insulin in the medicament delivery device 102. This will result in faster absorption and thus a shorter DIA. A final example recommendation 1610 is to avoid consuming certain foods, like foods with a high fat content and/or a high carbohydrates content. These foods may extend the user's DIA. The foods to avoid may be itemized or categorized in the recommendation 1610.

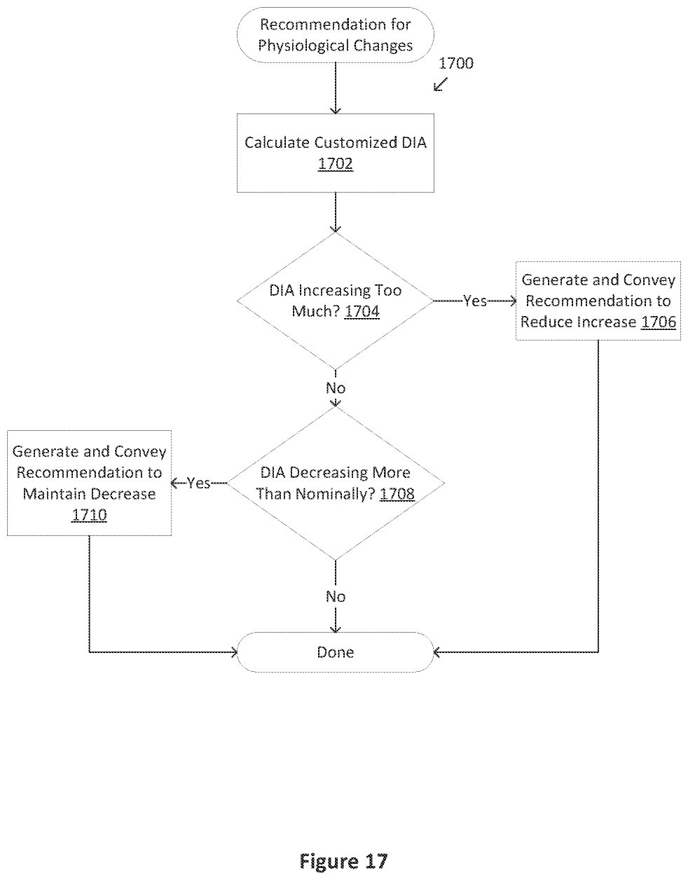

Recommendations may also be generated and conveyed when there is a significant deviation form initially calculated parameters. FIG. 17 depicts a flowchart 1700 of illustrative steps that may be performed in exemplary embodiments to generate recommendations for certain physiological changes that will improve the time in range for a user based on changes in the DIA of the user. At 1702, the customized DIA is calculated for a user on an ongoing basis, such as when an updated insulin decay curve is calculated. At 1704, a check is made whether the DIA is increasing too much. This may entail determining whether the DIA has exceeded a threshold or by calculating the rate of change of the DIA of the user and determining that the rate of change exceeds a threshold. At 1706, if it is determined that the DIA of the user is increasing too much, a recommendation to reduce the increase (e.g., to reverse the trend of the DIA from increasing) may be generated and sent.

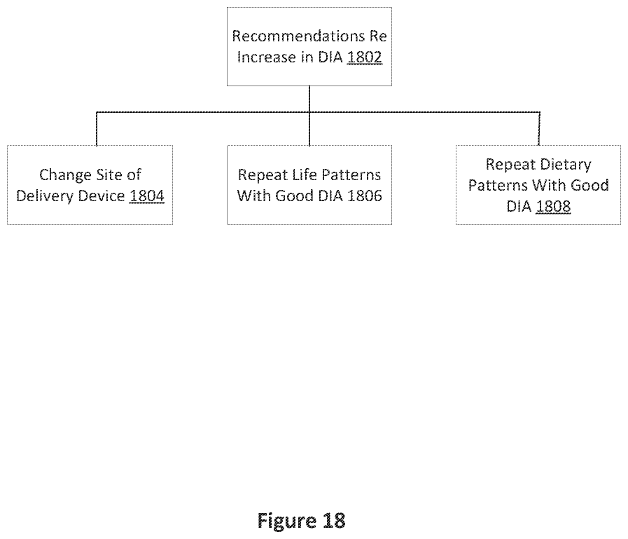

FIG. 18 depicts some illustrative recommendations 1802 that may be generated and conveyed at 1706. Recommendation 1804 suggests changing the site of the delivery device on the user. The current site may exhibit scarring or be a site that is subject to pressure or that does not absorb insulin as well. Another recommendation 1806 is to repeat life patterns where the user exhibited better DIA. This recommendation 1806 may reference a particular time frame or may identify behavior such as better sleep and exercise patterns. A further recommendation 1808 may be to exhibit dietary patterns that previously resulted in a better DIA. The recommendation 1808 may reference a particular period and/or may recommend certain foods while discouraging consumption of other foods.

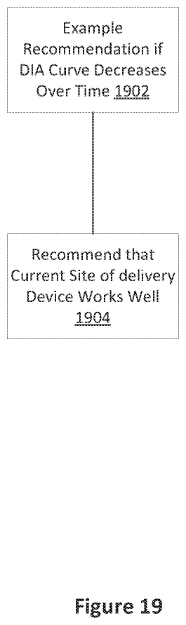

With reference again to FIG. 17, if, at 1704, it is determined that the DIA is not increasing too much, a check may be made at 1708 of whether the DIA of the user is decreasing more than a nominal amount. If so, this is desirable, and at 1710, a recommendation may be generated and conveyed to maintain the decrease. FIG. 19 depicts a process 1902 of making an example recommendation. The example recommendation 1904 may be to keep the medicament delivery device 202 at its current location on the user as it appears to be working well as evidenced by the decrease in DIA.

Other recommendations than those detailed above may be used. The example recommendations are intended to be illustrative and not limiting.

The IOB values used by the control application 216 or 220 in exemplary embodiments may be separated into a bolus contribution to the IOB (designated as IOB_{bol}) and the basal contribution to the IOB (designated as IOB_{AID}). Hence, IOB may viewed as the sum of IOB_{bol} and IOB_{AID}. The IOB_{bol} value may be useful in better constraining insulin deliveries following delivery of large insulin boluses than conventional systems that rely on a single IOB that does not take into account unique IOB_{bol} and IOB_{AID} values. As a user boluses more, the DIA decreases, according to methods described herein, so that the boluses do not unduly constrain AID insulin delivery. Moreover, DIA_{bol} may be shorter than DIA_{AID} to shorten the duration of time that the insulin delivery is constrained following large insulin boluses.

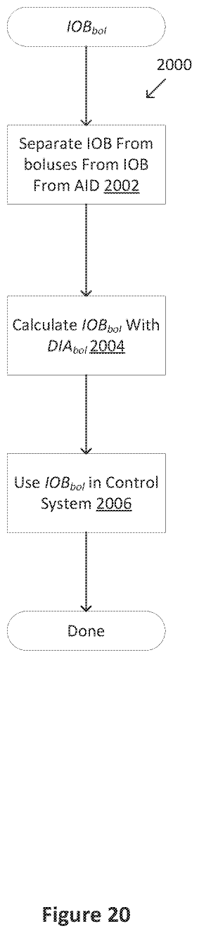

FIG. 20 depicts a flowchart 2000 of illustrative steps that may be performed in exemplary embodiments to calculate IOB_{bol} from DIA values that are particular to insulin boluses, designated as DIA_{bol}. At 2002, IOB_{bol} is separated from IOB_{AID}. At 2004, IOB_{bol} is calculated with DIA_{bol} and the bolus insulin deliveries for the past 12 cycles, such as by calculating:

$${IOB}_{bol} ( i ) = [ {I}_{bol} ( i ) - b ( i ) … {I}_{bol} ( i - {DIA}_{bol} \cdot 12 ) - b * i - {DIA}_{bol} \cdot 12 ) ] [ \begin{matrix}\begin{matrix}\begin{matrix}\begin{matrix}1 \\ 1 - \frac{1}{{DIA}_{bol} \cdot 12}\end{matrix} \\ 1 - \frac{2}{{DIA}_{bol} \cdot 12}\end{matrix} \\ ⋮\end{matrix} \\ 1 - \frac{i}{{DIA}_{bol} \cdot 12}\end{matrix} ] . \qquad ( Equation 5 )$$

At 2006, IOB_{bol} is used in the control system in predicting future glucose level values and constraining insulin bolus doses.

The DIA_{bol} value may be dynamically varied based on the recent insulin bolus delivery history. DIA_{bol} may be calculated as:

$${DIA}_{b o l} ( i ) = {DIA}_{max} - \frac{{Σ}_{k = 1}^{X \cdot 12} {I}_{b o l} ( i - k )}{TDI / 6} ( {DIA}_{max} - {DIA}_{min} ) \qquad ( Equation 6 )$$

where, over the past X hours (e.g., 6 hours), DIA. is the maximum permitted DIA value, DIAₘᵢₙ is the minimum permitted DIA value, I_{bol}(i−k) is the bolus at cycle i−k, i is the current cycle index (e.g., 12 five-minute cycles per hour), and TDI is total daily insulin for a user. The result is that the DIA_{bol} shortens as more insulin boluses have been delivered.

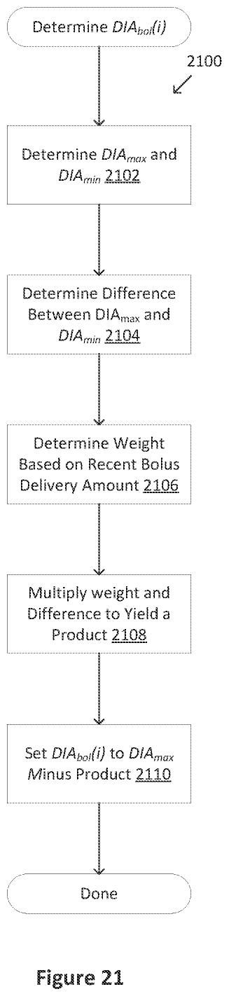

FIG. 21 depicts a flowchart 2100 of illustrative steps that may be performed in exemplary embodiments to determine DIA_{bol} (i) using Equation 6. At 2100, DIAₘₐₓ and DIAₘᵢₙ may be determined. DIA_{bol} may only assume values in the range defined by DIA. and DIAₘᵢₙ. At 2104, the difference between DIA. and DIA. is determined to identify the magnitude of the range. At 2106, a weight may be determined based on recent insulin bolus delivery amounts. The value Σₖ₌₁^{X·12} I_{bol}(i−k) sums the insulin bolus delivery amounts for the past X hours, where each cycle is 5 minutes in length (with 12 five-minute cycles per hour). X may be a suitable value such as 1, 3 or 6, for example. The summation is divided by TDI 16 in Equation 5 to represent ⅓ of the bolus insulin and represent a typical one time bolus need (i.e., one bolus per each of 3 daily meals), and assuming a 50:50 split between bolus and basal amounts that make up a user's TDI. At 2108, the weight and the difference are multiplied to produce a product. The product represents how much DIA_{bol}(i) is less than DIAₘₐₓ based on recent bolus deliveries. At 1210, DIA_{bol}(i) is set as the difference between DIAₘₐₓ and the product.

While exemplary embodiments have been described herein various changes in form and detail may be made without departing from the intended scope of the appended claims.

1. A medicament delivery system for delivering medicament to a user, comprising:
a non-transitory readable storage medium storing computer programming instructions;
a processor configured for executing the computer programming instructions, which cause the processor to:
create a model of analyte-medicament interactions that is customized for the user based on analyte level history and medicament delivery history;
determine a standard impulse response for the model;
determine, from the standard impulse response, an indication of how much medicament is on board over time for the user after a specified amount of medicament is delivered; and
use the indication in determining a dose of medicament for the user to be output by the medicament delivery system.

2. The medicament delivery system of claim 1, further comprising a reservoir for storing the medicament.

3. The medicament delivery system of claim 1, wherein the medicament is insulin, a glucagon like peptide-1 agonist, pramlintide, another glucose regulating agent, or combinations thereof.

4. The medicament delivery system of claim 1, wherein the analyte level is a glucose level.

5. The medicament delivery system of claim 1, wherein the indication is a curve, a function, an equation, a set of equations, a table, or a storage holding values that are indicative of how much medicament is on board for the user over time after the specified amount of medicament is delivered.

6. The medicament delivery system of claim 1, wherein the computer programming instructions also cause the processor to initiate delivery of the specified amount of medicament to the user.

7. The medicament delivery system of claim 1, wherein the medicament is insulin and wherein the model is a glucose prediction model that predicts a future glucose level value of the user based on earlier glucose level values for the user.

8. The medicament delivery system of claim 1, further comprising updating the indication to account for recent analyte level values for the user and using the updated indication in determining a next dose of medicament for the user to be output by the medicament delivery device.

9. The medicament delivery system of claim 1, wherein the indication of how much medicament is on board over time for the user after a specified amount of medicament is delivered is a nonlinear curve.

10. A medicament delivery system for delivering medicament to a user, comprising:
a non-transitory readable storage medium storing computer programming instructions;
a processor configured for executing the computer programming instructions, which cause the processor to:
determine an estimate of duration of insulin action for a user;
compare the estimate of duration of insulin action for the user to a threshold; and
where the estimate of duration of insulin action for the user exceeds a threshold, generate a notification of a recommendation to decrease the duration of insulin action for the user.

11. The medicament delivery system of claim 10, further comprising outputting the notification on a display device.

12. The medicament delivery system of claim 10, wherein the medicament delivery system includes an on body medical device that is secured to the user and wherein the recommendation recommends a change in medicament delivery device location on the user to reduce scarring.

13. The medicament delivery system of claim 10, wherein the medicament delivery device includes an on body medical device that is secured to the user and wherein the recommendation recommends a medicament delivery device location on the user that is typically not subject to pressure by contact.

14. The medicament delivery system of claim 10, wherein the recommendation recommends using faster acting insulin.

15. The medicament delivery system of claim 10, wherein the recommendation recommends avoiding ingestion of certain foods.

16. A medicament delivery system for delivering medicament to a user, comprising:
a non-transitory readable storage medium storing computer programming instructions;
a processor configured for executing the computer programming instructions, which cause the processor to:
determine an estimate of duration of insulin action for a user;
update the estimate of duration of insulin action for the user over time; and
where the estimate of duration of insulin action changes more than a threshold amount over time, generate a notification of a recommendation to reduce the change in the duration of insulin action for the user.

17. The medicament delivery system of claim 16, wherein the estimate of duration of insulin action is increasing, wherein the medicament delivery system includes a medicament delivery device, and wherein the recommendation is to change location of where the medicament delivery device is secured to the user.

18. The medicament delivery system of claim 16, wherein the estimate of duration of insulin action is increasing and wherein the recommendation is to modify diet patterns to previous diet patterns that resulted in acceptable glucose level control for the user.

19. The medicament delivery system of claim 16, wherein the estimate of duration of insulin action is decreasing, wherein the medicament delivery system includes a medicament delivery device, and wherein the recommendation is to keep a present location of where the medicament delivery device is secured to the user.

20. The medicament delivery system of claim 16, wherein the medicament delivery system includes an insulin pump adhered to the user.
